(19) United States
(12) Patent Application Publication
(10) Pub. No.: US 2023/0248042 A1
(43) Pub. Date: Aug. 10, 2023

(54) NUTRITIONAL COMPOSITION COMPRISING MIR-3184

(71) Applicant: SOCIETE DES PRODUITS NESTLE S.A., Vevey (CH)

(72) Inventors: FREDERIC RAYMOND, Chatel-St-Denis (CH); MOHAMED NABIL BOSCO, Epalinges (CH); LORANE TEXARI, Morges (CH); SOLENN PRUVOST, Cessy (FR); GREGORY LEFEBVRE, Vullierens (CH)

(21) Appl. No.: 18/015,108

(22) PCT Filed: Jul. 7, 2021
(86) PCT No.: PCT/EP2021/068778

§ 371 Date: Jan. 9, 2023

(30) Foreign Application Priority Data
Jul. 10, 2020 (EP) 20185309.0

(51) Int. Cl. A23L 33/13; A23L 33/00; C12N 15/113

(52) U.S. Cl. CPC A23L 33/13; A23L 33/40; C12N 15/113; C12N 2310/141

(57) ABSTRACT

A nutritional composition comprising miR-3184. miR-3184 or said nutritional composition for use as a medicament. Use of miR-3184 to modulate gene expression of one or more gene selected from Lipin 2 or Insulin Like Growth Factor 2. A method of producing said nutritional composition.

### FIELD OF THE INVENTION

The present invention relates to nutritional compositions comprising miR-3184. The present invention also relates to uses of miR-3184 and said nutritional compositions and methods of producing said nutritional compositions.

### BACKGROUND TO THE INVENTION

MicroRNAs (miRNAs) are small, non-coding RNAs around 17-25 nucleotides in length. They are regulatory RNA molecules that function to regulate the activity of specific mRNA targets and play important roles in a wide range of physiologic and pathologic processes. De-regulation of miRNA expression has been shown to have an impact on health and diseases (Wang et al. 2016, J. Cell Phys. 231:25-30).

Breast milk produced by mammals during lactation naturally contains miRNA. Milk miRNAs are found as free molecules but also packaged in microvesicles such as milk exosomes and fat globules. During lactation of a mother to infant, breast milk not only supplements nutrients to an infant, but also transfers miRNA between the mother and infant. This may promote healthy growth and development of the infant (Tome-Carneiro et al. 2018, Pharma. Res. 132:21-25).

While the beneficial effects of breast milk for the infant are known, not all infants have access to natural breast milk. Therefore, there is a need for nutritional compositions which mimic natural breast milk. In particular, there is a need to mimic the natural composition of breast milk at different stages of lactation as expression of these miRNA regulatory molecules correspond to the different growth and development needs of the infant over time after birth. In this regard, miRNA may be considered as an important component in breast milk during the different stages of lactation.

### SUMMARY OF THE INVENTION

The present inventors have found that miR-3184 is present in natural breast milk. In particular, the present inventors have found that expression of miR-3184 in natural breast milk increases between two weeks and three months postpartum. Further, miR-3184 might be implicated in the health and development of infants.

In one aspect the present invention provides a nutritional composition comprising miR-3184. The nutritional composition may be an infant formula, a fortifier, or a supplement. Preferably, the nutritional composition is an infant formula.

The miR-3184 may be present in a concentration of 0.1-10000 pmol/L, 0.1-1000 pmol/L, 1-1000 pmol/L, 10-1000 pmol/L, or 100-1000 pmol/L. Preferably, the miR-3184 is present in a concentration of 10-1000 pmol/L. More preferably, the miR-3184 is present in a concentration of 100-1000 pmol/L.

The nutritional composition may comprise one or more additional microRNAs selected from the list consisting of: let-7b, let-7c, miR-19b, miR-22, miR-24, miR-25, miR-29a, miR-30a, miR-92a, miR-99a, miR-100, miR-197, miR-30d, miR-181a, miR-181b, miR-205, miR-210, miR-221, miR-125b, miR-125a, miR-149, miR-193a, miR-320a, miR-200a, miR-99b, miR-130b, miR-30e, miR-375, miR-378a, miR-151a, miR-425, miR-484, miR-146b, miR-574, miR-652, miR-320c, miR-3141, let-7d, miR-196a, miR-187, miR-516a, miR-92b, and miR-3126. Preferably, the nutritional composition comprises one or more additional microRNAs selected from the list consisting of: let-7d, miR-196a, miR-187, miR-516a, miR-92b, and miR-3126. More preferably, the nutritional composition comprises miR-3126 and/or miR-3141. The one or more additional microRNAs may be present in a concentration of 0.1-10000 pmol/L, 0.1-1000 pmol/L, 1-1000 pmol/L, 10-1000 pmol/L, or 100-1000 pmol/L. Preferably, the one or more additional microRNAs are present in a concentration of 10-1000 pmol/L. More preferably, the one or more additional microRNAs are present in a concentration of 100-1000 pmol/L.

In another aspect the present invention provides a nutritional composition of the present invention for use as a medicament.

In another related aspect the present invention provides a nutritional composition of the present invention for use in protecting gastrointestinal health in a subject, promoting growth and development (e.g. neuronal and epidermal development) in a subject, or reducing the risk of a subject developing infections, lymphoproliferative disorders, allergic diseases (e.g. allergic asthma), or inflammatory diseases.

In another related aspect the present invention provides a method of feeding a subject comprising administering to the subject a nutritional composition of the present invention.

In another related aspect the present invention provides a method of protecting gastrointestinal health in a subject, promoting growth and development (e.g. neuronal and epidermal development) in a subject, or reducing the risk of a subject developing infections, lymphoproliferative disorders, allergic diseases (e.g. allergic asthma), or inflammatory diseases, comprising administering to the subject a nutritional composition of the present invention.

In another aspect the present invention provides miR-3184 for use as a medicament. The miR-3184 may be in a nutritional composition of the present invention.

In another related aspect the present invention provides miR-3184 for use in promoting healthy growth and development, for example in promoting long term metabolic health and/or preventing metabolic disorders later in life.

In another related aspect the present invention provides a method of feeding a subject comprising administering to the subject miR-3184.

In another related aspect the present invention provides a method of in promoting healthy growth and development in a subject, for example in promoting long term metabolic health and/or preventing metabolic disorders later in life in a subject. The miR-3184 may be in a nutritional composition of the present invention.

In preferred embodiments of these aspects of the present invention the subject is an infant. The infant may be 0-12 months old, 2-12 months old, 3-12 months old, 0-6 months old, 2-6 months old, or 3-6 months old. Preferably, the infant is 0-6 months old. More preferably, the infant is 2-6 months old. Most preferably, the infant is 3-6 months old.

In another aspect the present invention provides use of miR-3184 for providing a nutritional composition to mimic natural breast milk. The nutritional composition may be a nutritional composition according to the present invention.

In another aspect the present invention provides use of miR-3184 to modulate gene expression of one or more gene selected from: Lipin-2 and IGF2.

In another aspect the present invention provides a method of producing a nutritional composition of the present invention, comprising:
(i) providing a base nutritional composition; and
(ii) adding miR-3184 to the base nutritional composition to provide a nutritional of the present invention.

### DETAILED DESCRIPTION

Various preferred features and embodiments of the present invention will now be described by way of non-limiting examples.

It must be noted that as used herein and in the appended claims, the singular forms “a”, “an”, and “the” include plural referents unless the context clearly dictates otherwise.

The terms “comprising”, “comprises” and “comprised of” as used herein are synonymous with “including” or “includes”; or “containing” or “contains”, and are inclusive or open-ended and do not exclude additional, non-recited members, elements or steps. The terms “comprising”, “comprises” and “comprised of” also include the term “consisting of”.

As used herein the term “about” means approximately, in the region of, roughly, or around. When the term “about” is used in conjunction with a numerical value or range, it modifies that value or range by extending the boundaries above and below the numerical value(s) set forth. In general, the terms “about” and “approximately” are used herein to modify a numerical value(s) above and below the stated value(s) by 10%.

The publications discussed herein are provided solely for their disclosure prior to the filing date of the present application. Nothing herein is to be construed as an admission that such publications constitute prior art to the claims appended hereto.

This disclosure is not limited by the exemplary methods and materials disclosed herein, and any methods and materials similar or equivalent to those described herein can be used in the practice or testing of embodiments of this disclosure. Numeric ranges are inclusive of the numbers defining the range.

Nutritional Composition

In one aspect the present invention provides a nutritional composition comprising miR-3184.

According to the present invention, a “nutritional composition” means a composition which nourishes a subject. As used herein, the term “nutritional composition” does not include natural milk of human or animal origin, i.e. the nutritional composition is not natural human or animal milk. “Natural human milk” or “natural animal milk” refers to milk directly obtainable from a human or animal and does not encompass e.g. synthetic milk, infant formulas etc.

The nutritional composition of the present invention may comprise components derived from natural milk. For example, the nutritional composition of the present invention may comprise components derived from natural human milk and/or components derived from natural animal milk (e.g. bovine milk).

In a preferred embodiment of the invention, the nutritional composition contains miR-3184 as an active ingredient.

The nutritional composition is not particularly limited as long as it is suitable for administration (e.g. oral or intravenous administration). Examples of suitable nutritional compositions include foodstuffs, drinks, supplements, drug bases, and animal feeds.

Preferably, the nutritional composition according to the invention is suitable for infants. For example the nutritional composition may be an infant formula, a baby food, an infant cereal composition, a fortifier such as a human milk fortifier, or a supplement. Preferably, the nutritional composition is an infant formula, a fortifier, or a supplement.

In some embodiments the nutritional composition of the invention is a complete nutritional composition (fulfilling all or most of the nutritional needs of the subject). In other embodiments the nutrition composition is a supplement or a fortifier intended, for example, to supplement human milk or to supplement an infant formula.

The nutritional composition of the invention may be taken orally or intravenously, preferably orally.

The nutritional composition of the present invention can be in solid (e.g. powder), liquid or gelatinous form.

The nutritional composition according to the invention may be an enteral nutritional composition. An “enteral nutritional composition” is a foodstuff that involves the gastrointestinal tract for its administration.

The nutritional composition according to the invention may be a hypoallergenic nutritional composition. A “hypoallergenic” composition is a composition which is unlikely to cause allergic reactions.

The nutritional composition according to the invention may be prepared in any suitable manner.

Infant Formula

In preferred embodiments the nutritional composition is an infant formula.

The term “infant formula” may refer to a foodstuff intended for particular nutritional use by infants during the first year of life and satisfying by itself the nutritional requirements of this category of person, as defined in European Commission Regulation (EU) 2016/127 of 25 Sep. 2015. The term “infant formula” may also refer to a nutritional composition intended for infants and as defined in Codex Alimentarius (Codex STAN 72-1981).

The expression “infant formula” encompasses both “starter infant formula” and “follow-up formula” or “follow-on formula”. In one embodiment the infant formula is a starter infant formula. In one embodiment the infant formula is a follow-up formula or follow-on formula. A “follow-up formula” or “follow-on formula” is given from the 6th month onwards. Infant formula constitutes the principal liquid element in the progressively diversified diet of this category of person.

Infants can be fed solely with infant formula or the infant formula can be used as a complement of human milk.

The infant formula of the invention may be in the form of a powder or liquid.

The liquid may be, for example, a concentrated liquid infant formula or a ready-to-feed infant formula. The infant formula may be in the form of a reconstituted infant formula (i.e. a liquid infant formula that has been reconstituted from a powdered form). The concentrated liquid infant formula is preferably capable of being diluted into a liquid composition suitable for feeding an infant, for example by the addition of water.

In one embodiment, the infant formula is in a powdered form. The powder is capable of being reconstituted into a liquid composition suitable for feeding an infant, for example by the addition of water.

The infant formula may have an energy density of about 60-72 kcal per 100 mL, when formulated as instructed. Suitably, the infant formula may have an energy density of about 60-70 kcal per 100 mL, when formulated as instructed.

Fortifier

In other preferred embodiments the nutritional composition is a fortifier.

The term “fortifier” may refer to liquid or solid nutritional compositions suitable for mixing with breast milk or infant formula.

The fortifier may be a milk fortifier. The term “milk fortifier”, refers to any composition used to fortify or supplement either human breast milk or infant formula.

The fortifier may be, for example, 10-times concentrated, 15-times concentrated, 20-times concentrated, 25-times concentrated, 30-times concentrated, 35-times concentrated, 40-times concentrated, 45-times concentrated, 50-times concentrated, 60-times concentrated, 70-times concentrated, 80-times concentrated, 90-times concentrated, or 100-times concentrated, compared to the desired final concentration in the breast milk or infant formula.

Supplement

In other preferred embodiments the nutritional composition is a supplement.

A “supplement” or “dietary supplement” may be used to complement the nutrition of an individual (it is typically used as such but it might also be added to any kind of compositions intended to be ingested).

The supplement may be in the form of for example tablets, capsules, pastilles or a liquid. The supplement may further contain protective hydrocolloids (such as gums, proteins, modified starches), binders, film forming agents, encapsulating agents/materials, wall/shell materials, matrix compounds, coatings, emulsifiers, surface active agents, solubilizing agents (oils, fats, waxes, lecithins etc.), adsorbents, carriers, fillers, co-compounds, dispersing agents, wetting agents, processing aids (solvents), flowing agents, taste masking agents, weighting agents, jellifying agents and gel forming agents. The supplement may also contain conventional pharmaceutical additives and adjuvants, excipients and diluents, including, but not limited to, water, gelatine of any origin, vegetable gums, lignin-sulfonate, talc, sugars, starch, gum arabic, vegetable oils, polyalkylene glycols, flavouring agents, preservatives, stabilizers, emulsifying agents, buffers, lubricants, colorants, wetting agents, fillers, and the like.

Further, the supplement may contain an organic or inorganic carrier material suitable for oral or parenteral administration as well as vitamins, minerals trace elements and other micronutrients in accordance with the recommendations of Government bodies such as the USRDA.

When the nutritional composition is a supplement, it may be provided in the form of unit doses.

Pharmaceutical Composition

In some embodiments the nutritional composition is a pharmaceutical composition.

The form of the pharmaceutical preparation is not particularly limited, and examples include tablet, pill, powder, solution, suspension, emulsion, granule, capsule, syrup, and so forth. Additives widely used as pharmaceutical carriers for oral administration such as excipients, binders, disintegrating agents, lubricants, stabilizers, corrigents, diluents, and surfactants can be used.

Examples of suitable binders include starch, gelatin, natural sugars such as glucose, anhydrous lactose, free-flow lactose, beta-lactose, corn sweeteners, natural and gums, such as acacia, tragacanth or sodium alginate, carboxymethyl cellulose and polyethylene glycol.

Examples of suitable lubricants include sodium oleate, sodium stearate, magnesium stearate, sodium benzoate, sodium acetate, sodium chloride and the like.

Components

The nutritional composition of the invention may contain a protein source, a carbohydrate source and/or a lipid source. In some embodiments however, especially if the nutritional composition of the invention is a supplement or a fortifier, there may be only lipids (or a lipid source).

Protein

The nutritional composition according to the present invention may contain a protein source.

The protein may be present in the nutritional composition of the present invention in any suitable amount. For example, the protein content of the nutritional composition of the invention may be in the range of from 1.6 to 3 g per 100 kcal, especially when the nutritional composition is an infant formula. In some embodiments, especially when the nutritional composition is intended for premature infants, the protein amount can be between 2.4 and 4 g/100 kcal or more than 3.6 g/100 kcal. In some other embodiments the protein amount can be below 2.0 g per 100 kcal, e.g. between 1.8 to 2 g/100 kcal, or in an amount below 1.8 g per 100 kcal.

The protein source may be any protein source which is suitable for use in a nutritional composition. Protein sources based on, for example, whey, casein and mixtures thereof may be used as well as protein sources based on soy. As far as whey proteins are concerned, the protein source may be based on acid whey or sweet whey or mixtures thereof and may include alpha-lactalbumin and beta-lactoglobulin in any desired proportions. In some embodiments the protein source is whey predominant (i.e. more than 50% of proteins are coming from whey proteins, such as 60% or 70%).

The proteins may be intact or hydrolysed or a mixture of intact and hydrolysed proteins.

By the term “intact” is meant that the main part of the proteins are intact, i.e. the molecular structure is not altered, for example at least 80% of the proteins are not altered, such as at least 85% of the proteins are not altered, preferably at least 90% of the proteins are not altered, even more preferably at least 95% of the proteins are not altered, such as at least 98% of the proteins are not altered. In a particular embodiment, 100% of the proteins are not altered.

The term “hydrolysed” means in the context of the present invention a protein which has been hydrolysed or broken down into its component amino acids.

The proteins may be either fully or partially hydrolysed. It may be desirable to supply partially hydrolysed proteins (degree of hydrolysis between 2 and 20), for example for infants or young children believed to be at risk of developing cow's milk allergy. The degree of hydrolysis (DH) of the protein can be between 2 and 20, or between 8 and 40, or between 20 and 60 or between 20 and 80 or more than 10, 20, 40, 60, 80 or 90. In one embodiment of the invention at least 70% of the proteins are hydrolysed, preferably at least 80% of the proteins are hydrolysed, such as at least 85% of the proteins are hydrolysed, even more preferably at least 90% of the proteins are hydrolysed, such as at least 95% of the proteins are hydrolysed, particularly at least 98% of the proteins are hydrolysed. In a particular embodiment, 100% of the proteins are hydrolysed.

If hydrolysed proteins are required, the hydrolysis process may be carried out as desired and as is known in the art. For example, whey protein hydrolysates may be prepared by enzymatically hydrolysing the whey fraction in one or more steps. If the whey fraction used as the starting material is substantially lactose free, it is found that the protein suffers much less lysine blockage during the hydrolysis process. This enables the extent of lysine blockage to be reduced from about 15% by weight of total lysine to less than about 10% by weight of lysine; for example about 7% by weight of lysine which greatly improves the nutritional quality of the protein source.

Carbohydrate

The nutritional composition according to the present invention may contain a carbohydrate source.

The carbohydrate may be present in the nutritional composition of the present invention in any suitable amount. For example, the carbohydrate content of the nutritional composition of the invention may be in the range 9-14 g carbohydrate per 100 kcal, especially when the nutritional composition is an infant formula.

The carbohydrate source may be any carbohydrate source which is suitable for use in a nutritional composition. Some suitable carbohydrate sources include lactose, sucrose, saccharose, maltodextrin, starch and mixtures thereof may be used.

Fat

The nutritional composition according to the present invention may contain a source of lipids (fat).

The fat may be present in the nutritional composition of the present invention in any suitable amount. For example, the fat content of the nutritional composition of the invention may be in the range 4.0-6.0 g fat per 100 kcal, especially when the nutritional composition is an infant formula.

Example fats for use in the nutritional composition of the invention include sunflower oil, low erucic acid rapeseed oil, safflower oil, canola oil, olive oil, coconut oil, palm kernel oil, soybean oil, fish oil, palm oleic, high oleic sunflower oil and high oleic safflower oil, and microbial fermentation oil containing long chain, polyunsaturated fatty acids.

The fat may also be in the form of fractions derived from these oils, such as palm olein, medium chain triglycerides (MCT) and esters of fatty acids such as arachidonic acid, linoleic acid, palmitic acid, stearic acid, docosahexaeonic acid, linolenic acid, oleic acid, lauric acid, capric acid, caprylic acid, caproic acid, and the like.

Further example fats include structured lipids (i.e. lipids that are modified chemically or enzymatically in order to change their structure). Preferably, the structured lipids are sn2 structured lipids, for example comprising triglycerides having an elevated level of palmitic acid at the sn2 position of the triglyceride. Structured lipids may be added or may be omitted.

Oils containing high quantities of preformed arachidonic acid (ARA) and/or docosahexaenoic acid (DHA), such as fish oils or microbial oils, may be added.

Long chain polyunsaturated fatty acids, such as dihomo-γ-linolenic acid, arachidonic acid (ARA), eicosapentaenoic acid and docosahexaenoic acid (DHA), may also be added.

The essential fatty acids linoleic and α-linolenic acid may also be added, as well small amounts of oils containing high quantities of preformed arachidonic acid and docosahexaenoic acid such as fish oils or microbial oils. The fat source may have a ratio of n-6 to n-3 fatty acids of about 5:1 to about 15:1; for example about 8:1 to about 10:1.

Further Components

The nutritional composition of the invention may also contain any suitable vitamins and minerals.

For example, the nutritional composition of the invention may contain all vitamins and minerals understood to be essential in the daily diet and in nutritionally significant amounts. Minimum requirements have been established for certain vitamins and minerals. Examples of minerals, vitamins and other nutrients optionally present in the nutritional composition of the invention include vitamin A, vitamin B1, vitamin B2, vitamin B6, vitamin B12, vitamin E, vitamin K, vitamin C, vitamin D, folic acid, inositol, niacin, biotin, pantothenic acid, choline, calcium, phosphorous, iodine, iron, magnesium, copper, zinc, manganese, chlorine, potassium, sodium, selenium, chromium, molybdenum, taurine, and L-carnitine. Minerals are usually added in salt form. The presence and amounts of specific minerals and other vitamins will vary depending on the intended population.

The nutritional composition of the invention may contain emulsifiers and stabilisers such as soy, lecithin, citric acid esters of mono- and diglycerides, and the like.

The nutritional composition of the invention may also contain one or more carotenoid. In some particular embodiments of the invention, the nutritional composition of the invention does not comprise any carotenoids.

The nutritional composition of the invention may also contain other substances which may have a beneficial effect such as lactoferrin, osteopontin, TGFbeta, sIgA, glutamine, nucleotides, nucleosides, and the like.

The nutritional composition of the invention can further comprise at least one non-digestible oligosaccharide (e.g. prebiotics).

Examples of prebiotics may be fructooligosaccharide, galactooligosaccharide, acidic oligosaccharides, human milk oligosaccharide (HMO), or bovine's milk oligosaccharide (BMO) like cow's milk oligosaccharide (CMO) such as “CMOS-GOS”. Some examples are N-acetylated oligosaccharides, sialylated oligosaccharides, fucosylated oligosaccharides and any mixtures thereof.

The nutritional composition of the present invention can further comprise at least one probiotic (or probiotic strain), such as a probiotic bacterial strain. The term “probiotic” refers to microbial cell preparations or components of microbial cells with beneficial effects on the health or well-being of the host. In particular, probiotics may improve gut barrier function.

Preferred probiotics are those which as a whole are safe, are L(+) lactic acid producing cultures and have acceptable shelf-life for products that are required to remain stable and effective for up to 24 months.

Examples of probiotic micro-organisms for use in the nutritional composition of the present invention include yeasts, such as Saccharomyces, Debaromyces, Candida, Pichia and Torulopsis; and bacteria, such as the genera Bifidobacterium, Bacteroides, Clostridium, Fusobacterium, Melissococcus, Propionibacterium, Streptococcus, Enterococcus, Lactococcus, Staphylococcus, Peptostrepococcus, Bacillus, Pediococcus, Micrococcus, Leuconostoc, Weissella, Aerococcus, Oenococcus and Lactobacillus.

Specific examples of suitable probiotic microorganisms are: Saccharomyces cereviseae, Bacillus coagulans, Bacillus licheniformis, Bacillus subtilis, Bifidobacterium bifidum, Bifidobacterium infantis, Bifidobacterium longum, Enterococcus faecium, Enterococcus faecalis, Lactobacillus acidophilus, Lactobacillus alimentarius, Lactobacillus casei subsp. casei, Lactobacillus casei Shirota, Lactobacillus curvatus, Lactobacillus delbrueckii subsp. lactis, Lactobacillus farciminus, Lactobacillus gasseri, Lactobacillus helveticus, Lactobacillus johnsonii, Lactobacillus rhamnosus (Lactobacillus GG), Lactobacillus sake, Lactococcus lactis, Micrococcus varians, Pediococcus acidilactici, Pediococcus pentosaceus, Pediococcus acidilactici, Pediococcus halophilus, Streptococcus faecalis, Streptococcus thermophilus, Staphylococcus carnosus and Staphylococcus xylosus.

microRNAs

MicroRNAs (miRNAs) are small, non-coding RNAs around 17-25 nucleotides in length. They are regulatory RNA molecules that function to regulate the activity of specific mRNA targets.

Mature miRNA is denoted by the prefix “miR” followed by a dash and a number. Uncapitalized “mir-” refers to the pre-miRNA and the pri-miRNA. “MIR” refers to the human gene that encodes the miRNA.

In the present invention, mature miRNA is preferably used. However, the present invention may also be carried out using pre-miRNA and/or pri-miRNA. Mature miRNA can be obtained by digesting pre-miRNA and/or pri-miRNA with a Dicer enzyme (e.g. Dicer1) or the like, which occur naturally in humans and animals. Accordingly, whilst mature miRNA is generally referred to (e.g. miR-3184), pre-miRNA and/or pri-miRNA (e.g. pri-miR-3184) could also be used in the present invention (e.g. in combination with mature miRNA or instead of mature miRNA). Thus, the mature miRNA referred to herein may be replaced with corresponding pre-miRNA and/or pri-miRNA.

Species of origin is designated with a three-letter prefix, e.g., hsa-miR-124 is a human (Homo sapiens) miRNA and oar-miR-124 is a sheep (Ovis aries) miRNA.

When two mature microRNAs originate from opposite arms of the same pre-miRNA and are found in roughly similar amounts, they are denoted with a -3p or -5p suffix. If the mature microRNA found from one arm of the hairpin is much more abundant than that found from the other arm, an asterisk following the name indicates the mature species found at low levels from the opposite arm of a hairpin. For example, miR-124 and miR-124* share a pre-miRNA hairpin, but much more miR-124 is found in the cell.

miRNA sequences have been deposited in miRBase database (http://www.mirbase.org/). The miRBase database is a searchable database of published miRNA sequences and annotation.

miR-3184

The present inventors have found that miR-3184 is present in natural breast milk. In particular, the present inventors have found that expression of miR-3184 in natural breast milk increases between two weeks and three months postpartum.

Accordingly, in one aspect the present invention provides use of miR-3184 for providing a nutritional composition. In particular, the present invention provides use of miR-3184 for providing a nutritional composition to mimic natural breast milk (e.g. human breast milk). The nutritional composition may be a nutritional composition according to the present invention.

miR-3184 is also known as MicroRNA 3184 and has accession number M10014226.

The miR-3184 for use in the present invention may comprise or consist of miR-3184-5p and/or miR-3184-3p. Preferably, the miR-3184 comprises miR-3184-3p For example, at least 50%, at least 60%, at least 70%, at least 80%, at least 90%, at least 95%, at least 99% or 100% of the miR-3184 may be miR-3184-3p. More preferably, the miR-3184 consists of miR-3184-3p.

The miR-3184 for use in the present invention may be human miR-3184, i.e. hsa-miR-3184.

The miR-3184 for use in the present invention may comprise or consist of hsa-miR-3184-5p and/or hsa-miR-3184-3p. Preferably, the miR-3184 comprises hsa-miR-3184-3p. For example, at least 50%, at least 60%, at least 70%, at least 80%, at least 90%, at least 95%, at least 99% or 100% of the miR-3184 may be hsa-miR-3184-5p. More preferably, the miR-3184 consists of hsa-miR-3184-5p.

An illustrative sequence for hsa-mir-3184 (i.e. the pre-miRNA from which hsa-miR-3184 is derived) is shown below as SEQ ID NO: 1. The miR-3184 for use in the present invention may be derived from pre-miRNA comprising or consisting of a sequence which has at least 20%, at least 30%, at least 50%, at least 60%, at least 70%, at least 80%, at least 85%, at least 90%, at least 95%, or 100% identity to SEQ ID NO: 1. Preferably, the miR-3184 for use in the present invention is derived from pre-miRNA comprising of consisting of a sequence which has at least 95% identity to SEQ ID NO: 1. More preferably, the miR-3184 for use in the present invention is derived from pre-miRNA comprising of consisting of a sequence according to SEQ ID NO: 1.

SEQ ID NO: 1-illustrative hsa-mir-3184 sequence
AAGCAAGACUGAGGGGCCUCAGACCGAGCUUUUGGAAAAUAGAAAAGU
CUCGCUCUCUGCCCCUCAGCCUAACUU

An illustrative sequence for hsa-miR-3184-5p (i.e. mature miRNA) is shown below as SEQ ID NO: 2. The miR-3184 for use in the present invention may comprise or consist of a sequence which has at least 80%, at least 85%, at least 90%, at least 95%, or 100% identity to SEQ ID NO: 2. Preferably, the miR-3184 for use in the present invention comprises or consists of a sequence which has at least 95% identity to SEQ ID NO: 2. More preferably, the miR-3184 for use in the present invention comprises or consists of a sequence according to SEQ ID NO: 2.

SEQ ID NO: 2-illustrative hsa-miR-3184-5p sequence
UGAGGGGCCUCAGACCGAGCUUUU

An illustrative sequence for hsa-miR-3184-3p (i.e. mature miRNA) is shown below as SEQ ID NO: 3. The miR-3184 for use in the present invention may comprise or consist of a sequence which has at least 80%, at least 85%, at least 90%, at least 95%, at least 98%, at least 99%, or 100% identity to SEQ ID NO: 3. Preferably, the miR-3184 for use in the present invention comprises or consists of a sequence which has at least 95% identity to SEQ ID NO: 2. More preferably, the miR-3184 for use in the present invention comprises or consists of a sequence according to SEQ ID NO: 3.

SEQ ID NO: 3-illustrative hsa-miR-3184-3p sequence
AAAGUCUCGCUCUCUGCCCCUCA

The miR-3184 may be present in the nutritional composition of the present invention in a concentration of 0.1-10000 pmol/L, 0.1-1000 pmol/L, 1-1000 pmol/L, 10-1000 pmol/L, or 100-1000 pmol/L, especially when the nutritional composition is an infant formula. Preferably, the miR-3184 is present in the nutritional composition of the present invention in a concentration of 10-1000 pmol/L, especially when the nutritional composition is an infant formula. More preferably, the miR-3184 is present in the nutritional composition of the present invention in a concentration of 100-1000 pmol/L, especially when the nutritional composition is an infant formula. The miR-3184 may be present in the nutritional composition of the present invention such that the concentration is about the same as in natural breast milk.

When the nutritional composition is a fortifier, the miR-3184 may be present in the fortifier such that after mixing with breast milk or infant formula the concentration of miR-3184 is 0.1-10000 pmol/L, 0.1-1000 pmol/L, 1-1000 pmol/L, 10-1000 pmol/L, or 100-1000 pmol/L. Preferably, the miR-3184 is present in the fortifier such that after mixing with breast milk or infant formula the concentration of miR-3184 is 10-1000 pmol/L. More preferably, the miR-3184 is present in the fortifier such that after mixing with breast milk or infant formula the concentration of miR-3184 is 100-1000 pmol/L. The miR-3184 may be present in the fortifier such that after mixing with breast milk or infant formula the concentration of miR-3184 is about the same as in natural breast milk.

When the nutritional composition is a supplement, the miR-3184 may be present in the supplement such that a unit dose is provided. Thus, the supplement may provide a miR-3184 dose equivalent to 50-250 ml, 100-250 ml, 150-250 ml, or about 100 ml, or about 200 ml of natural breast milk. For example, miR-3184 may be present in the supplement in an amount of 0.02-2000 pmol, 0.02-200 pmol, 0.2-200 pmol, 2-200 pmol, or 20-200 pmol. Preferably, miR-3184 is present in the supplement in an amount of 2-200 pmol. More preferably, miR-3184 is present in the supplement in an amount of 20-200 pmol.

The nutritional composition of the present invention preferably comprises hsa-miR-3184-3p in a concentration of 10-1000 pmol/L, especially when the nutritional composition is an infant formula. The nutritional composition of the present invention more preferably comprises hsa-miR-3184-3p in a concentration of 100-1000 pmol/L, especially when the nutritional composition is an infant formula.

When the nutritional composition is in a powder form capable of being reconstituted into a liquid composition, the concentration of miR-3184 is based on the reconstituted liquid composition.

Other miRNAs

The nutritional composition of the present invention may comprise one or more microRNAs in addition to miR-3184.

For example, the nutritional composition may comprise one or more additional microRNAs which are abundant in natural breast milk. Suitable additional miRNAs which are abundant in natural breast milk include: let-7b, let-7c, miR-19b, miR-22, miR-24, miR-25, miR-29a, miR-30a, miR-92a, miR-99a, miR-100, miR-197, miR-30d, miR-181a, miR-181b, miR-205, miR-210, miR-221, miR-125b, miR-125a, miR-149, miR-193a, miR-320a, miR-200a, miR-99b, miR-130b, miR-30e, miR-375, miR-378a, miR-151a, miR-425, miR-484, miR-146b, miR-574, miR-652, miR-320c, miR-3141, let-7d, miR-196a, miR-187, miR-516a, miR-92b, and miR-3126.

The nutritional composition may comprise one or more additional microRNAs selected from: let-7b, let-7c, miR-19b, miR-22, miR-24, miR-25, miR-29a, miR-30a, miR-92a, miR-99a, miR-100, miR-197, miR-30d, miR-181a, miR-181b, miR-205, miR-210, miR-221, miR-125b, miR-125a, miR-149, miR-193a, miR-320a, miR-200a, miR-99b, miR-130b, miR-30e, miR-375, miR-378a, miR-151a, miR-425, miR-484, miR-146b, miR-574, miR-652, miR-320c, and miR-3141.

The nutritional composition may comprise one or more additional microRNAs selected from: let-7d, miR-196a, miR-187, miR-516a, miR-92b, and miR-3126.

In one embodiment the nutritional composition comprises miR-3126 and/or miR-3141. In another embodiment the nutritional composition comprises miR-3126.

The one or more additional microRNAs for use in the present invention may comprise or consist of human or bovine microRNAs. Preferably the one or more additional microRNAs are human microRNAs.

The one or more additional microRNAs for use in the present invention may comprise or consist of both mature microRNAs originating from opposite arms of the same pre-miRNA. Preferably, the one or more additional microRNAs for use in the present invention comprise or consist of a mature microRNA from one arm of the hairpin, for example the mature microRNA which is most abundant in natural breast milk.

The nutritional composition may comprise one or more additional microRNAs selected from: hsa-let-7b-5p, hsa-let-7c-5p, hsa-miR-19b-3p, hsa-miR-22-3p, hsa-miR-24-3p, hsa-miR-25-3p, hsa-miR-29a-3p, hsa-miR-30a-5p, hsa-miR-92a-3p, hsa-miR-99a-5p, hsa-miR-100-5p, hsa-miR-197-3p, hsa-miR-30d-5p, hsa-miR-181a-5p, hsa-miR-181b-5p, hsa-miR-205-5p, hsa-miR-210-3p, hsa-miR-221-3p, hsa-miR-125b-5p, hsa-miR-125a-5p, hsa-miR-149-3p, hsa-miR-193a-5p, hsa-miR-193a-3p, hsa-miR-320a, hsa-miR-200a-3p, hsa-miR-99b-5p, hsa-miR-130b-3p, hsa-miR-30e-5p, hsa-miR-375, hsa-miR-378a-3p, hsa-miR-151a-3p, hsa-miR-425-5p, hsa-miR-484, hsa-miR-146b-5p, hsa-miR-574-5p, hsa-miR-652-3p, hsa-miR-320c, hsa-miR-3141, hsa-let-7d-3p, hsa-miR-196a-5p, hsa-miR-187-5p, hsa-miR-516a-5p, hsa-miR-92b-5p, hsa-miR-3126-5p, bta-let-7b, bta-let-7c, bta-miR-19b, bta-miR-22-3p, bta-miR-24-3p, bta-miR-25, bta-miR-29a, bta-miR-30a-5p, bta-miR-92a, bta-miR-99a-5p, bta-miR-100, bta-miR-197, bta-miR-30d, bta-miR-181a, bta-miR-181b, bta-miR-205, bta-miR-210, bta-miR-221, bta-miR-125b, bta-miR-125a, bta-miR-149-3p, bta-miR-193a-5p, bta-miR-193a-3p, bta-miR-320a, bta-miR-200a, bta-miR-99b, bta-miR-130b, bta-miR-30e-5p, bta-miR-375, bta-miR-378, bta-miR-151-3p, bta-miR-425-5p, bta-miR-484, bta-miR-146b, bta-miR-574, bta-miR-652, bta-miR-320b, bta-miR-3141, bta-let-7d, bta-miR-196a-5p, bta-miR-187-5p, and bta-miR-92b-5p.

The nutritional composition may comprise one or more additional microRNAs selected from: hsa-let-7b-5p, hsa-let-7c-5p, hsa-miR-19b-3p, hsa-miR-22-3p, hsa-miR-24-3p, hsa-miR-25-3p, hsa-miR-29a-3p, hsa-miR-30a-5p, hsa-miR-92a-3p, hsa-miR-99a-5p, hsa-miR-100-5p, hsa-miR-197-3p, hsa-miR-30d-5p, hsa-miR-181a-5p, hsa-miR-181b-5p, hsa-miR-205-5p, hsa-miR-210-3p, hsa-miR-221-3p, hsa-miR-125b-5p, hsa-miR-125a-5p, hsa-miR-149-3p, hsa-miR-193a-5p, hsa-miR-193a-3p, hsa-miR-320a, hsa-miR-200a-3p, hsa-miR-99b-5p, hsa-miR-130b-3p, hsa-miR-30e-5p, hsa-miR-375, hsa-miR-378a-3p, hsa-miR-151a-3p, hsa-miR-425-5p, hsa-miR-484, hsa-miR-146b-5p, hsa-miR-574-5p, hsa-miR-652-3p, hsa-miR-320c, hsa-miR-3141, hsa-let-7d-3p, hsa-miR-196a-5p, hsa-miR-187-5p, hsa-miR-516a-5p, hsa-miR-92b-5p, hsa-miR-3126-5p.

The nutritional composition may comprise one or more additional microRNAs selected from: hsa-let-7b-5p, hsa-let-7c-5p, hsa-miR-19b-3p, hsa-miR-22-3p, hsa-miR-24-3p, hsa-miR-25-3p, hsa-miR-29a-3p, hsa-miR-30a-5p, hsa-miR-92a-3p, hsa-miR-99a-5p, hsa-miR-100-5p, hsa-miR-197-3p, hsa-miR-30d-5p, hsa-miR-181a-5p, hsa-miR-181b-5p, hsa-miR-205-5p, hsa-miR-210-3p, hsa-miR-221-3p, hsa-miR-125b-5p, hsa-miR-125a-5p, hsa-miR-149-3p, hsa-miR-193a-5p, hsa-miR-193a-3p, hsa-miR-320a, hsa-miR-200a-3p, hsa-miR-99b-5p, hsa-miR-130b-3p, hsa-miR-30e-5p, hsa-miR-375, hsa-miR-378a-3p, hsa-miR-151a-3p, hsa-miR-425-5p, hsa-miR-484, hsa-miR-146b-5p, hsa-miR-574-5p, hsa-miR-652-3p, hsa-miR-320c, hsa-miR-3141, bta-let-7b, bta-let-7c, bta-miR-19b, bta-miR-22-3p, bta-miR-24-3p, bta-miR-25, bta-miR-29a, bta-miR-30a-5p, bta-miR-92a, bta-miR-99a-5p, bta-miR-100, bta-miR-197, bta-miR-30d, bta-miR-181a, bta-miR-181b, bta-miR-205, bta-miR-210, bta-miR-221, bta-miR-125b, bta-miR-125a, bta-miR-149-3p, bta-miR-193a-5p, bta-miR-193a-3p, bta-miR-320a, bta-miR-200a, bta-miR-99b, bta-miR-130b, bta-miR-30e-5p, bta-miR-375, bta-miR-378, bta-miR-151-3p, bta-miR-425-5p, bta-miR-484, bta-miR-146b, bta-miR-574, bta-miR-652, bta-miR-320b, and bta-miR-3141.

The nutritional composition may comprise one or more additional microRNAs selected from: hsa-let-7b-5p, hsa-let-7c-5p, hsa-miR-19b-3p, hsa-miR-22-3p, hsa-miR-24-3p, hsa-miR-25-3p, hsa-miR-29a-3p, hsa-miR-30a-5p, hsa-miR-92a-3p, hsa-miR-99a-5p, hsa-miR-100-5p, hsa-miR-197-3p, hsa-miR-30d-5p, hsa-miR-181a-5p, hsa-miR-181b-5p, hsa-miR-205-5p, hsa-miR-210-3p, hsa-miR-221-3p, hsa-miR-125b-5p, hsa-miR-125a-5p, hsa-miR-149-3p, hsa-miR-193a-5p, hsa-miR-193a-3p, hsa-miR-320a, hsa-miR-200a-3p, hsa-miR-99b-5p, hsa-miR-130b-3p, hsa-miR-30e-5p, hsa-miR-375, hsa-miR-378a-3p, hsa-miR-151a-3p, hsa-miR-425-5p, hsa-miR-484, hsa-miR-146b-5p, hsa-miR-574-5p, hsa-miR-652-3p, hsa-miR-320c, and hsa-miR-3141.

The nutritional composition may comprise one or more additional microRNAs selected from: hsa-let-7d-3p, hsa-miR-196a-5p, hsa-miR-187-5p, hsa-miR-516a-5p, hsa-miR-92b-5p, hsa-miR-3126-5p, bta-let-7d, bta-miR-196a-5p, bta-miR-187-5p, and bta-miR-92b-5p.

The nutritional composition may comprise one or more additional microRNAs selected from: hsa-let-7d-3p, hsa-miR-196a-5p, hsa-miR-187-5p, hsa-miR-516a-5p, hsa-miR-92b-5p, and hsa-miR-3126-5p.

In one embodiment, the nutritional composition comprises hsa-miR-3126-5p and/or hsa-miR-3141. In another embodiment, the nutritional composition comprises hsa-miR-3126-5p.

Illustrative sequences for suitable additional microRNAs are shown in Table 1 below.

TABLE 1
Illustrative microRNA sequences

| | Illustrative | | Illustrative |
|---|---|---|---|
| Human miRNA | sequence | Bovine miRNA | sequence |
| hsa-let-7b-5p | UGAGGUAGUAGGUUGUG | bta-let-7b | UGAGGUAGUAGGUUGUG |
| | UGGUU | | UGGUU |
| | (SEQ ID NO: 4) | | (SEQ ID NO: 49) |
| hsa-let-7c-5p | UGAGGUAGUAGGUUGUA | bta-let-7c | UGAGGUAGUAGGUUGUA |
| | UGGUU | | UGGUU |
| | (SEQ ID NO: 5) | | (SEQ ID NO: 50) |
| hsa-miR-19b-3p | UGUGCAAAUCCAUGCAAA | bta-miR-19b | UGUGCAAAUCCAUGCAAA |
| | ACUGA | | ACUGA |
| | (SEQ ID NO: 6) | | (SEQ ID NO: 51) |
| hsa-miR-22-3p | AAGCUGCCAGUUGAAGA | bta-miR-22-3p | AAGCUGCCAGUUGAAGAA |
| | ACUGU | | CUG |
| | (SEQ ID NO: 7) | | (SEQ ID NO: 52) |
| hsa-miR-24-3p | UGGCUCAGUUCAGCAGG | bta-miR-24-3p | UGGCUCAGUUCAGCAGG |
| | AACAG | | AACAG |
| | (SEQ ID NO: 8) | | (SEQ ID NO: 53) |
| hsa-miR-25-3p | CAUUGCACUUGUCUCGG | bta-miR-25 | CAUUGCACUUGUCUCGG |
| | UCUGA | | UCUGA |
| | (SEQ ID NO: 9) | | (SEQ ID NO: 54) |
| hsa-miR-29a-3p | UAGCACCAUCUGAAAUC | bta-miR-29a | CUAGCACCAUCUGAAAUC |
| | GGUUA | | GGUUA |
| | (SEQ ID NO: 10) | | (SEQ ID NO: 55) |
| hsa-miR-30a-5p | UGUAAACAUCCUCGACU | bta-miR-30a-5p | UGUAAACAUCCUCGACUG |
| | GGAAG | | GAAGCU |
| | (SEQ ID NO: 11) | | (SEQ ID NO: 56) |
| hsa-miR-92a-3p | UAUUGCACUUGUCCCGG | bta-miR-92a | UAUUGCACUUGUCCCGG |
| | CCUGU | | CCUGU |
| | (SEQ ID NO: 12) | | (SEQ ID NO: 57) |
| hsa-miR-99a-5p | AACCCGUAGAUCCGAUC | bta-miR-99a-5p | AACCCGUAGAUCCGAUCU |
| | UUGUG | | UGU |
| | (SEQ ID NO: 13) | | (SEQ ID NO: 58) |
| hsa-miR-100-5p | AACCCGUAGAUCCGAAC | bta-miR-100 | AACCCGUAGAUCCGAACU |
| | UUGUG | | UGUG |
| | (SEQ ID NO: 14) | | (SEQ ID NO: 59) |
| hsa-miR-197-3p | UUCACCACCUUCUCCAC | bta-miR-197 | UUCACCACCUUCUCCACC |
| | CCAGC | | CAGC |
| | (SEQ ID NO: 15) | | (SEQ ID NO: 60) |
| hsa-miR-30d-5p | UGUAAACAUCCCCGACU | bta-miR-30d | UGUAAACAUCCCCGACUG |
| | GGAAG | | GAAGCU |
| | (SEQ ID NO: 16) | | (SEQ ID NO: 61) |
| hsa-miR-181a-5p | AACAUUCAACGCUGUCG | bta-miR-181a | AACAUUCAACGCUGUCGG |
| | GUGAGU | | UGAGUU |
| | (SEQ ID NO: 17) | | (SEQ ID NO: 62) |
| hsa-miR-181b-5p | AACAUUCAUUGCUGUCG | bta-miR-181b | AACAUUCAUUGCUGUCGG |
| | GUGGGU | | UGGGUU |
| | (SEQ ID NO: 18) | | (SEQ ID NO: 63) |
| hsa-miR-205-5p | UCCUUCAUUCCACCGGA | bta-miR-205 | UCCUUCAUUCCACCGGAG |
| | GUCUG | | UCUG |
| | (SEQ ID NO: 19) | | (SEQ ID NO: 64) |
| hsa-miR-210-3p | CUGUGCGUGUGACAGCG | bta-miR-210 | ACUGUGCGUGUGACAGC |
| | GCUGA | | GGCUGA |
| | (SEQ ID NO: 20) | | (SEQ ID NO: 65) |
| hsa-miR-221-3p | AGCUACAUUGUCUGCUG | bta-miR-221 | AGCUACAUUGUCUGCUG |
| | GGUUUC | | GGUUU |
| | (SEQ ID NO: 21) | | (SEQ ID NO: 66) |
| hsa-miR-125b-5p | UCCCUGAGACCCUAACU | bta-miR-125b | UCCCUGAGACCCUAACUU |
| | UGUGA | | GUGA |
| | (SEQ ID NO: 22) | | (SEQ ID NO: 67) |
| hsa-miR-125a-5p | UCCCUGAGACCCUUUAA | bta-miR-125a | UCCCUGAGACCCUUUAAC |
| | CCUGUGA | | CUGUG |
| | (SEQ ID NO: 23) | | (SEQ ID NO: 68) |
| hsa-miR-149-3p | AGGGAGGGACGGGGGCU | bta-miR-149-3p | GAGGGAGGGACGGGGGC |
| | GUGC | | UGUGC |
| | (SEQ ID NO: 24) | | (SEQ ID NO: 69) |
| hsa-miR-193a-5p | UGGGUCUUUGCGGGCGA | bta-miR-193a-5p | UGGGUCUUUGCGGGCGA |
| | GAUGA | | GAUGA |
| | (SEQ ID NO: 25) | | (SEQ ID NO: 70) |
| hsa-miR-193a-3p | AACUGGCCUACAAAGUC | bta-miR-193a-3p | AACUGGCCUACAAAGUCC |
| | CCAGU | | CAGU |
| | (SEQ ID NO: 26) | | (SEQ ID NO: 71) |
| hsa-miR-320a | AAAAGCUGGGUUGAGAG | bta-miR-320a | AAAAGCUGGGUUGAGAG |
| | GGCGA | | GGCGA |
| | (SEQ ID NO: 27) | | (SEQ ID NO: 72) |
| hsa-miR-200a-3p | UAACACUGUCUGGUAAC | bta-miR-200a | UAACACUGUCUGGUAACG |
| | GAUGU | | AUGUU |
| | (SEQ ID NO: 28) | | (SEQ ID NO: 73) |
| hsa-miR-99b-5p | CACCCGUAGAACCGACC | bta-miR-99b | CACCCGUAGAACCGACCU |
| | UUGCG | | UGCG |
| | (SEQ ID NO: 29) | | (SEQ ID NO: 74) |
| hsa-miR-130b-3p | CAGUGCAAUGAUGAAAG | bta-miR-130b | CAGUGCAAUGAUGAAAGG |
| | GGCAU | | GCAU |
| | (SEQ ID NO: 30) | | (SEQ ID NO: 75) |
| hsa-miR-30e-5p | UGUAAACAUCCUUGACU | bta-miR-30e-5p | UGUAAACAUCCUUGACUG |
| | GGAAG | | GAAGCU |
| | (SEQ ID NO: 31) | | (SEQ ID NO: 76) |
| hsa-miR-375 | UUUGUUCGUUCGGCUCG | bta-miR-375 | UUUUGUUCGUUCGGCUC |
| | CGUGA | | GCGUGA |
| | (SEQ ID NO: 32) | | (SEQ ID NO: 77) |
| hsa-miR-378a-3p | ACUGGACUUGGAGUCAG | bta-miR-378 | ACUGGACUUGGAGUCAGA |
| | AAGGC | | AGGC |
| | (SEQ ID NO: 33) | | (SEQ ID NO: 78) |
| hsa-miR-151a-3p | CUAGACUGAAGCUCCUU | bta-miR-151-3p | CUAGACUGAAGCUCCUUG |
| | GAGG | | AGG |
| | (SEQ ID NO: 34) | | (SEQ ID NO: 79) |
| hsa-miR-425-5p | AAUGACACGAUCACUCCC | bta-miR-425-5p | AUGACACGAUCACUCCCG |
| | GUUGA | | UUGA |
| | (SEQ ID NO: 35) | | (SEQ ID NO: 80) |
| hsa-miR-484 | UCAGGCUCAGUCCCCUC | bta-miR-484 | UCAGGCUCAGUCCCCUCC |
| | CCGAU | | CGAU |
| | (SEQ ID NO: 36) | | (SEQ ID NO: 81) |
| hsa-miR-146b-5p | UGAGAACUGAAUUCCAUA | bta-miR-146b | UGAGAACUGAAUUCCAUA |
| | GGCU | | GGCUGU |
| | (SEQ ID NO: 37) | | (SEQ ID NO: 82) |
| hsa-miR-574-5p | UGAGUGUGUGUGUGUGA | bta-miR-574 | UGAGUGUGUGUGUGUGA |
| | GUGUGU | | GUGUGUG |
| | (SEQ ID NO: 38) | | (SEQ ID NO: 83) |
| hsa-miR-652-3p | AAUGGCGCCACUAGGGU | bta-miR-652 | AAUGGCGCCACUAGGGU |
| | UGUG | | UGUG |
| | (SEQ ID NO: 39) | | (SEQ ID NO: 84) |
| hsa-miR-320c | AAAAGCUGGGUUGAGAG | bta-miR-320b | AGCUGGGUUGAGAGGGU |
| | GGU | | GGU |
| | (SEQ ID NO: 40) | | (SEQ ID NO: 85) |
| hsa-miR-3141 | GAGGGCGGGUGGAGGAG | bta-miR-3141 | GAGGGCGGGUGGAGGAG |
| | GA | | G |
| | (SEQ ID NO: 41) | | (SEQ ID NO: 86) |
| hsa-miR-4478 | GAGGCUGAGCUGAGGAG | NA | NA |
| | (SEQ ID NO: 42) | | |
| hsa-let-7d-3p | CUAUACGACCUGCUGCC | bta-let-7d | AGAGGUAGUAGGUUGCA |
| | UUUCU | | UAGUU |
| | (SEQ ID NO: 43) | | (SEQ ID NO: 87) |
| hsa-miR-196a-5p | UAGGUAGUUUCAUGUUG | bta-miR-196a-5p | UAGGUAGUUUCAUGUUG |
| | UUGGG | | UUGGG |
| | (SEQ ID NO: 44) | | (SEQ ID NO: 88) |
| hsa-miR-187-5p | GGCUACAACACAGGACC | bta-miR-187-5p | UCGUGUCUUGUGUUGCA |
| | CGGGC | | GCCGG |
| | (SEQ ID NO: 45) | | (SEQ ID NO: 89) |
| hsa-miR-516a-5p | UUCUCGAGGAAAGAAGC | NA | NA |
| | ACUUUC | | |
| | (SEQ ID NO: 46) | | |
| hsa-miR-92b-5p | AGGGACGGGACGCGGUG | bta-miR-92b-5p | UAUUGCACUCGUCCCGG |
| | CAGUG | | CCUCC |
| | (SEQ ID NO: 47) | | (SEQ ID NO: 90) |
| hsa-miR-3126-5p | UGAGGGACAGAUGCCAG | | |
| | AAGCA | | |
| | (SEQ ID NO: 48) | | |

The nutritional composition may comprise one or more additional microRNAs with at least 80%, at least 85%, at least 90%, at least 95%, or 100% identity to any one of SEQ ID NOs: 4-90. The nutritional composition may comprise one or more additional microRNAs which have a sequence according to SEQ ID NOs: 4-90 given in table 1.

The nutritional composition may comprise one or more additional microRNAs with at least 80%, at least 85%, at least 90%, at least 95%, or 100% identity to any one of SEQ ID NOs: 4-42 or 49-86. The nutritional composition may comprise one or more additional microRNAs which have a sequence according to SEQ ID NOs: 4-42 or 49-86 given in table 1.

The nutritional composition may comprise one or more additional microRNAs with at least 80%, at least 85%, at least 90%, at least 95%, or 100% identity to any one of SEQ ID NOs: 4-42. The nutritional composition may comprise one or more additional microRNAs which have a sequence according to SEQ ID NOs: 4-42 given in table 1.

The nutritional composition may comprise one or more additional microRNAs with at least 80%, at least 85%, at least 90%, at least 95%, or 100% identity to any one of SEQ ID NOs: 43-48 or 87-90. The nutritional composition may comprise one or more additional microRNAs which have a sequence according to SEQ ID NOs: 43-48 or 87-90 given in table 1.

The nutritional composition may comprise one or more additional microRNAs with at least 80%, at least 85%, at least 90%, at least 95%, or 100% identity to any one of SEQ ID NOs: 43-48. The nutritional composition may comprise one or more additional microRNAs which have a sequence according to SEQ ID NOs: 43-48 given in table 1.

The nutritional composition may comprise a microRNA with at least 80%, at least 85%, at least 90%, at least 95%, or 100% identity to SEQ ID NO: 41 or 48. Preferably, the nutritional composition comprises a microRNA which has a sequence according to SEQ ID NO: 41 or 48. More preferably, the nutritional composition comprises a microRNA which has a sequence according to SEQ ID NO: 48.

Each of the one or more additional microRNAs may be present in the nutritional composition of the present invention in a concentration of 0.1-10000 pmol/L, 0.1-1000 pmol/L, 1-1000 pmol/L, 10-1000 pmol/L, or 100-1000 pmol/L, especially when the nutritional composition is an infant formula. Preferably, each of the one or more additional microRNAs are present in the nutritional composition of the present invention in a concentration of 10-1000 pmol/L, especially when the nutritional composition is an infant formula. More preferably, each of the one or more additional microRNAs are present in the nutritional composition of the present invention in a concentration of 100-1000 pmol/L, especially when the nutritional composition is an infant formula. The one or more additional microRNAs may be present in the nutritional composition of the present invention such that the concentration of each of the one or more additional microRNAs is about the same as in natural breast milk.

When the nutritional composition is a fortifier, each of the one or more additional microRNAs may be present in the fortifier such that after mixing with breast milk or infant formula the concentration of each of the one or more additional microRNAs is 0.1-10000 pmol/L, 0.1-1000 pmol/L, 1-1000 pmol/L, 10-1000 pmol/L, or 100-1000 pmol/L. Preferably, each of the one or more additional microRNAs are present in the fortifier such that after mixing with breast milk or infant formula the concentration of each of the one or more additional microRNAs is 10-1000 pmol/L. More preferably, the each of the one or more additional microRNAs is present in the fortifier such that after mixing with breast milk or infant formula the concentration of each of the one or more additional microRNAs is 100-1000 pmol/L. The one or more additional microRNAs may be present in the fortifier such that after mixing with breast milk or infant formula the concentration of each of the one or more additional microRNAs is about the same as in natural breast milk.

When the nutritional composition is a supplement, each of the one or more additional microRNAs may be present in the fortifier such that supplement such that a unit dose of each microRNA is provided. Thus, the supplement may provide a dose equivalent to 50-250 ml, 100-250 ml, 150-250 ml, or about 100 ml, or about 200 ml of natural breast milk. For example, each of the one or more additional microRNAs may be present in the supplement in an amount of 0.02-2000 nmol, 0.02-200 nmol, 0.2-200 nmol, 2-200 nmol, or 20-200 nmol. Preferably, each of the one or more additional microRNAs is present in the supplement in an amount of 2-200 nmol. More preferably, each of the one or more additional microRNAs is present in the supplement in an amount of 20-200 nmol.

When the nutritional composition is in a powder form capable of being reconstituted into a liquid composition, the concentration of each of the one or more additional microRNAs is based on the reconstituted liquid composition.

Preparation of miRNAs

miRNAs for use in the present invention can be obtained by any suitable method known in the art.

miRNAs may be prepared synthetically or isolated from a body fluid.

Mature miRNA can be prepared synthetically by preparing a partially double-stranded RNA as a precursor of miRNA (pre-miRNA), and digesting it with a Dicer enzyme. As the Dicer enzyme, commercially available enzymes can be used. Double-stranded RNA (e.g. pre-miRNA) can be prepared by, for example, a RNA polymerase reaction using a double-stranded DNA having a complementary sequence as a template. Double-stranded DNA can be prepared by amplification based on PCR using a chromosomal DNA of mammal as a template and primers designed so as to be able to amplify the sequence of miRNA.

Further, miRNA can also be prepared by chemical synthesis. That is, miRNA can be obtained by synthesizing a sense strand and an antisense strand and annealing them.

miRNA may be isolated from colostrum or breast milk. miRNA may be isolated from colostrum or breast milk of bovine origin.

Variants and Fragments

In addition to the miRNAs mentioned herein, the present invention also encompasses the use of variants and fragments thereof.

The term “variant” as used herein means an miRNA having a certain homology with the wild type miRNA sequence or the SEQ ID NOs disclosed herein. The term “homology” can be equated with “identity”.

A variant miRNA sequence may include a nucleotide sequence which may be at least 50%, at least 55%, at least 65%, at least 70%, at least 75%, at least 80%, at least 85% or at least 90% identical, preferably at least 95%, at least 96%, at least 97%, at least 98%, or at least 99% identical to the subject miRNA sequence. Typically, the miRNA variants will have similar chemical properties/functions, e.g. effect the same gene regulation, as the subject miRNA sequence. Although homology can also be considered in terms of similarity (i.e. amino acid residues having similar chemical properties/functions), in the context of the present invention it is preferred to express homology in terms of sequence identity.

Identity comparisons can be conducted by eye or, more usually, with the aid of readily available sequence comparison programs. These commercially available computer programs can calculate percentage homology or identity between two or more sequences.

Percentage homology may be calculated over contiguous sequences, i.e. one sequence is aligned with the other sequence and each nucleotide in one sequence is directly compared with the corresponding nucleotide in the other sequence, one residue at a time. This is called an “ungapped” alignment. Typically, such ungapped alignments are performed only over a relatively short number of residues.

Although this is a very simple and consistent method, most sequence comparison methods are designed to produce optimal alignments that take into consideration possible insertions and deletions without penalising unduly the overall homology score. This is achieved by inserting “gaps” in the sequence alignment to try to maximise local homology.

However, these more complex methods assign “gap penalties” to each gap that occurs in the alignment so that, for the same number of identical nucleotides, a sequence alignment with as few gaps as possible, reflecting higher relatedness between the two compared sequences, will achieve a higher score than one with many gaps. “Affine gap costs” are typically used that charge a relatively high cost for the existence of a gap and a smaller penalty for each subsequent residue in the gap. This is the most commonly used gap scoring system. High gap penalties will of course produce optimised alignments with fewer gaps. Most alignment programs allow the gap penalties to be modified. However, it is preferred to use the default values when using such software for sequence comparisons.

Calculation of maximum percentage homology therefore firstly requires the production of an optimal alignment, taking into consideration gap penalties. A suitable computer program for carrying out such an alignment is the GCG Wisconsin Bestfit package (University of Wisconsin, U.S.A.; Devereux et al. (1984) Nucleic Acids Res. 12: 387). Examples of other software that can perform sequence comparisons include, but are not limited to, the BLAST package (see Ausubel et al. (1999) ibid—Ch. 18), FASTA (Atschul et al. (1990) J. Mol. Biol. 403-410) and the GENEWORKS suite of comparison tools. Both BLAST and FASTA are available for offline and online searching (see Ausubel et al. (1999) ibid, pages 7-58 to 7-60). However, for some applications, it is preferred to use the GCG Bestfit program. Another tool, called BLAST 2 Sequences is also available for comparing nucleotide sequences (see FEMS Microbiol. Lett. (1999) 174: 247-50; FEMS Microbiol. Lett. (1999) 177: 187-8).

Although the final percentage homology can be measured in terms of identity, the alignment process itself is typically not based on an all-or-nothing pair comparison. Instead, a scaled similarity score matrix is generally used that assigns scores to each pairwise comparison based on chemical similarity or evolutionary distance. An example of such a matrix commonly used is the BLOSUM62 matrix—the default matrix for the BLAST suite of programs. GCG Wisconsin programs generally use either the public default values or a custom symbol comparison table if supplied (see the user manual for further details). For some applications, it is preferred to use the public default values for the GCG package, or in the case of other software, the default matrix, such as BLOSUM62. Suitably, the percentage identity is determined across the entirety of the reference and/or the query sequence.

Once the software has produced an optimal alignment, it is possible to calculate percentage homology, preferably percentage sequence identity. The software typically does this as part of the sequence comparison and generates a numerical result.

“Fragments” typically refers to a selected region of the miRNA that is of interest functionally. “Fragment” thus refers to a miRNA sequence that is a portion of a full-length miRNA.

Such variants and fragments may be prepared using standard techniques.

Regulation of Gene Expression

The inventors have shown that miR-3184 modulates the expression of genes including the gene encoding Lipin 2 and IGF2.

Accordingly, in one aspect the present invention provides use of miR-3184 to modulate gene expression of one or more gene selected from: Lipin 2 and IGF2.

Lipin 2

The inventors have shown that miR-3184 significantly increases expression of Lipin 2.

Lipin 2 (UniProt Q92539) is also known as Phosphatidate phosphatase LPIN2.

Lipin 2 plays important roles in controlling the metabolism of fatty acids at different levels. Lipin 2 acts as a magnesium-dependent phosphatidate phosphatase enzyme which catalyzes the conversion of phosphatidic acid to diacylglycerol during triglyceride, phosphatidylcholine and phosphatidylethanolamine biosynthesis in the reticulum endoplasmic membrane. Lipin 2 acts also as a nuclear transcriptional coactivator for PPARGC1A to modulate lipid metabolism.

Lipin 2 may also have an anti-inflammatory role. Lipin-2 regulates NLRP3 inflammasome by affecting P2X7 receptor activation. Mutations in human LPIN2 produce a disease known as Majeed syndrome. 2—deficient mice exhibit increased sensitivity to high lipopolysaccharide doses. (Lordén, G., et al., 2016. Journal of Experimental Medicine, 214(2), pp. 511-528).

Insulin Like Growth Factor 2 (IGF2)

The inventors have shown that miR-3184 significantly increases expression of IGF2.

IFG2 (UniProtKB P01344) is also known as Insulin Like Growth Factor II (IGF2).

The insulin-like growth factors possess growth-promoting activity. Major fetal growth hormone in mammals. Plays a key role in regulating fetoplacental development. IGF-II is influenced by placental lactogen. Also involved in tissue differentiation.

Insulin-like growth factor 2 (IGF-2) is necessary for adequate human growth. Overexpression of the IGF2 gene is associated with fetal overgrowth and may play a role in the intrauterine programming of adipose tissue (https://www.karger.com/Article/FullText/443500). There is a body of evidence on the relationship of IGF-2 with weight and adiposity in infants.

IGF-1 and IGF-2 are peptides primarily secreted by the liver and in utero by placenta. Disruption of IGF1 and IGF2 genes in knockout mice studies led to reductions in birth weight. Heterozygous loss of the IGF2 gene led to a 60% reduction in birth weight compared to wild-type mice, suggesting that IGF-2 is a larger contributor to intrauterine growth.

IGF-2 signal transduction occurs through binding at the type 1 IGF receptor (IGF-1R) and insulin receptors. The IGF-1R has a higher affinity for IGF-2 than the insulin receptors.

Uses of miR-3184 and the Nutritional Composition

In one aspect the invention provides miR-3184 for use as a medicament. In a related aspect the invention provides for use of miR-3184 for the manufacture of a medicament. In a related aspect the invention provides a method of treatment comprising administering miR-3184.

In another aspect the invention provides the nutritional composition of the invention for use as a medicament. In a related aspect the invention provides for use of the nutritional composition of the invention for the manufacture of a medicament. In a related aspect the invention provides a method of treatment comprising administering the nutritional composition of the invention.

Healthy Growth and Development

As described above, the inventors have shown that miR-3184 significantly increases expression of Lipin-2 and IGF2. As above described such genes have a role in regulation of lipid metabolism and adequate growth, respectively.

In one aspect the invention provides miR-3184 for use in promoting healthy growth and/or development. In a related aspect the invention provides for use of miR-3184 for the manufacture of a medicament for promoting growth and/or development. In a related aspect the invention provides a method of promoting healthy growth and/or development of a subject comprising administering miR-3184 to the subject.

In one aspect the invention provides the nutritional composition of the invention for use in promoting healthy growth and/or development. In a related aspect the invention provides the nutritional composition of the invention for the manufacture of a medicament for promoting healthy growth and/or development. In a related aspect the invention provides a method of promoting healthy growth and/or development of a subject comprising administering the nutritional composition of the invention to the subject.

In one aspect the invention provides use of miR-3184 in promoting healthy growth and/or development. In another aspect the invention provides use of the nutritional composition of the invention in promoting healthy growth and/or development.

In one embodiment of the present invention, promotion of healthy growth is promotion of long term metabolic health and/or prevention of metabolic health disorders later in life, such as for example obesity, type 2 diabetes, insulin resistance.

The Subject

miR-3184 and/or the nutritional composition of the invention may be administered to any subject in need thereof.

Preferably the subject is an infant or a young child. More preferably the subject is an infant. Thus, miR-3184 and/or the nutritional composition of the invention may be administered to an infant.

“Infant” means a child under the age of 12 months. Thus, miR-3184 and/or the nutritional composition of the invention may be administered to an infant, wherein the infant is 0-12 months of age. In one embodiment, the infant is 0-6 months of age.

In one embodiment the infant is 2-12 months of age, preferably 2-6 months of age.

In one embodiment the infant is 3-12 months of age, preferably 3-6 months of age.

Accordingly, in some embodiments miR-3184 and/or the nutritional composition of the invention are administered to an infant, wherein the infant is 3-6 months of age.

In some embodiments the infant or young child is a preterm infant or young child. A “preterm” or “premature” means an infant or young child who was not born at term. Generally it refers to an infant or young child born prior 36 weeks of gestation.

In some embodiments the infant or young child was born by C-section or was vaginally delivered.

Methods of Manufacture

The nutritional composition of the present invention may be prepared by any suitable method known in the art.

For example, a nutritional composition may be prepared by blending together a protein source, a carbohydrate source and a fat source in appropriate proportions. If used, emulsifiers may be included at this point. Vitamins and minerals may be added at this point but they may be added later to avoid thermal degradation. Any lipophilic vitamins, emulsifiers and the like may be dissolved into the fat source prior to blending. Water, preferably water which has been subjected to reverse osmosis, may then be mixed in to form a liquid mixture. The temperature of the water is conveniently in the range between about 50° C. and about 80° C. to aid dispersal of the ingredients. Commercially available liquefiers may be used to form the liquid mixture.

The miRNA may be added at this point, especially if the final product is to have a liquid form. If the final product is to be a powder, they may likewise be added at this stage if desired. Alternatively the miRNA may be added later to avoid thermal degradation.

The liquid mixture may then be homogenised, for example in two stages.

The liquid mixture may then be thermally treated to reduce bacterial loads, by rapidly heating the liquid mixture to a temperature in the range between about 80° C. and about 150° C. for a duration between about 5 seconds and about 5 minutes, for example. This may be carried out by means of steam injection, an autoclave or a heat exchanger, for example a plate heat exchanger.

Then, the liquid mixture may be cooled to between about 60° C. and about 85° C. for example by flash cooling. The liquid mixture may then be again homogenised, for example in two stages between about 10 MPa and about 30 MPa in the first stage and between about 2 MPa and about 10 MPa in the second stage. The homogenised mixture may then be further cooled to add any heat sensitive components, such as vitamins and minerals. The pH and solids content of the homogenised mixture are conveniently adjusted at this point.

If the final product is to be a powder, the homogenised mixture is transferred to a suitable drying apparatus such as a spray dryer or freeze dryer and converted to powder. The powder should have a moisture content of less than about 5% by weight. The miRNA may also or alternatively be added at this stage by dry-mixing or by blending and the mixture is spray-dried or freeze-dried.

If a liquid composition is preferred, the homogenised mixture may be sterilised then aseptically filled into suitable containers or may be first filled into the containers and then retorted.

### EXAMPLES

### Example 1—Longitudinal Profile and Analysis of miRNAs in Human Breast Milk Sample Processing

Milk samples were collected from 44 individuals at three time points: V2, corresponding to about two weeks postpartum; V4, corresponding to about 2 months postpartum; and V5, corresponding to about 3 months postpartum.

Whole milk samples were first thawed on ice. miRNA profiling was performed with the HTG EdgeSeq platform, using the HTG EdgeSeq miRNA Whole Transcriptome Assay (HTG Molecular Diagnostics).

30 μL of whole milk was added to 30 μL of Plasma Lysis Buffer (ratio 1:1) complemented by 6 μL of Proteinase K (1/10). After a 3-hour incubation at 50° C. at 600 rpm, 26 μL of the mixture were transferred to the HTG platform for miRNA capture. miRNA-target specific probes hybridized against their corresponding miRNAs and protected them from S1 nuclease digestion. Base hydrolysis eliminated the captured miRNAs and the probes were conserved for indexing and sequencing library preparation.

Once the capture was completed, each sample was tagged with sequencing indexes and specific sample barcodes by a PCR step. The PCR mixture consisted of 15 μL OneTaq® Hot Start 2X Master Mix GC Buffer (NEB), 3 μL of each HTG tag primers (forward and reverse), 3 μL of the sample capture, and 6 μL of nuclease free water. Following a denaturation step of 4 min at 95° C., 20 PCR cycles were performed, consisting of a denaturation step of 15 s at 95° C., an annealing step of 45 s at 56° C. and an extension step of 45 s at 68° C. The PCR reaction was completed by a final step of 10 min at 68° C. PCR products were purified with CleanNGS beads (ratio 1:8) on SciClone NGS WorkStation (Perkin Elmer), and visualized on a LabChip GX High Sensitivity (Perkin Elmer) for the determination of their nucleic acids concentrations.

Purified PCR products were pooled by 24 samples on a liquid handling robotic platform at an equimolar concentration of 2 nM in 100 μL final volume. Pools were purified a second time with CleanNGS beads (ratio 1:8) and quantified with Qubit to precisely adjust the final concentration to 2 nM. The Illumina MiSeq sequencer was loaded with 20 pM of library spiked with 5% (v/v) PhiX. A MiSeq 150V3 kit was used for the sequencing of each pool.

Sequencing raw data (named “reads”) were parsed using the HTG Processor software (version 2), and the mapped miRNA reads were further analyzed.

Data Analysis

6 samples set outside the 95% confidence interval ellipse and were therefore considered as outliers. As a consequence, these samples were excluded from the analysis dataset.

miRNAs which were below detection threshold in more than 50 samples were excluded. An offset value of 1 to the expression level was set prior to the log 2 transformation. From the Count per Million (CPM) distribution, we estimated the detection threshold to 7 CPMs. In total, 685 miRNAs were measured.

Three analytical approaches were used to analyse the dataset. To optimize the selection of miRNA expression profiles and decrease the number of false positive results, the intersection between the three analytical approaches were kept as the best miRNA profiles. These three approaches are described below.

Longitudinal Model with Quantile Normalization

In the first approach, the dataset was normalized using the quantile method.

The dataset was modelled with a linear mixed model. In order to correct as well for inter-donor variability during both time points, and at baseline, a random term for the donor was added to the model. To correct for gender profile discrepancies observed for some miRNA during both time points and at baseline, a gender random effect was added to the model. To correct for the sequencing run effect, a sequencing run random effect was added to the model. Eventually because we were performing more than 600 tests, we needed to correct for multiple testing. A typical 5% false discovery rate threshold was then applied to the results.

miRNA_{Expr}=Time+(Donor)+(Gender)+(SeqRun)+Error

Longitudinal Model with TMM Normalization

In the second approach, the dataset was normalized using the TMM method. The dataset was next modelled and tested with the same model as for the first approach.

Classical Differential Expression Between V5 and V2

In the third approach, the dataset was normalized using the quantile method but modelled with a simpler generalized linear model. This method does not account for the longitudinal design of the study and the non-independency of the samples belonging to the same donor.

miRNA_{Expr}=Time+SeqRun+Error

A typical 5% false discovery threshold was then applied to the results.

Results

Eleven dynamic miRNAs were identified with either an increasing or a decreasing expression during lactation. These are shown in Tables 1 and 2 below.

Thirty-eight miRNAs were identified which were highly and stably expressed during lactation. These are shown in Table 3 below.

TABLE 1
seven miRNA with
increased expression during lactation

| miRNA | |
|---|---|
| human ID | miRNA human sequence |
| has-let-7d-3p | CUAUACGACCUGCUGCCUUUCU |
| has-miR- | UAGGUAGUUUCAUGUUGUUGGG |
| 196a-5p | |
| has-miR-187- | GGCUACAACACAGGACCCGGGC |
| 5p | |
| has-miR- | UUCUCGAGGAAAGAAGCACUUUC |
| 516a-5p | |
| has-miR-92b- | AGGGACGGGACGCGGUGCAGUG |
| 5p | |
| has-miR- | UGAGGGACAGAUGCCAGAAGCA |
| 3126-5p | |
| has-miR- | AAAGUCUCGCUCUCUGCCCCUCA |
| 3184-3p | |

TABLE 2
four miRNA with
decreased expression during lactation

| miRNA | |
|---|---|
| human ID | miRNA human sequence |
| has-miR-34a- | UGGCAGUGUCUUAGCUGGUUGU |
| 5p | |
| has-miR- | UCCCUGAGACCCUAACUUGUGA |
| 125b-5p | |
| has-miR- | UGAGAACUGAAUUCCAUGGGUU |
| 146a-5p | |
| has-miR- | ACUCGGCGUGGCGUCGGUCGUG |
| 1307-3p | |

TABLE 3
thirty-eight highly and stably expressed miRNA

| miRNA human ID | miRNA human sequence |
|---|---|
| has-let-7b-5p | UGAGGUAGUAGGUUGUGUGGUU |
| has-let-7c-5p | UGAGGUAGUAGGUUGUAUGGUU |
| has-miR-19b-3p | UGUGCAAAUCCAUGCAAAACUGA |
| has-miR-22-3p | AAGCUGCCAGUUGAAGAACUGU |
| has-miR-24-3p | UGGCUCAGUUCAGCAGGAACAG |
| has-miR-25-3p | CAUUGCACUUGUCUCGGUCUGA |
| has-miR-29a-3p | UAGCACCAUCUGAAAUCGGUUA |
| has-miR-30a-5p | UGUAAACAUCCUCGACUGGAAG |
| has-miR-92a-3p | UAUUGCACUUGUCCCGGCCUGU |
| has-miR-99a-5p | AACCCGUAGAUCCGAUCUUGUG |
| has-miR-100-5p | AACCCGUAGAUCCGAACUUGUG |
| has-miR-197-3p | UUCACCACCUUCUCCACCCAGC |
| has-miR-30d-5p | UGUAAACAUCCCCGACUGGAAG |
| has-miR-181a-5p | AACAUUCAACGCUGUCGGUGAGU |
| has-miR-181b-5p | AACAUUCAUUGCUGUCGGUGGGU |
| has-miR-205-5p | UCCUUCAUUCCACCGGAGUCUG |
| has-miR-210-3p | CUGUGCGUGUGACAGCGGCUGA |
| has-miR-221-3p | AGCUACAUUGUCUGCUGGGUUUC |
| has-miR-125b-5p | UCCCUGAGACCCUAACUUGUGA |
| has-miR-125a-5p | UCCCUGAGACCCUUUAACCUGUGA |
| has-miR-149-3p | AGGGAGGGACGGGGGCUGUGC |
| has-miR-193a-5p | UGGGUCUUUGCGGGCGAGAUGA |
| has-miR-193a-3p | AACUGGCCUACAAAGUCCCAGU |
| has-miR-320a | AAAAGCUGGGUUGAGAGGGCGA |
| has-miR-200a-3p | UAACACUGUCUGGUAACGAUGU |
| has-miR-99b-5p | CACCCGUAGAACCGACCUUGCG |
| has-miR-130b-3p | CAGUGCAAUGAUGAAAGGGCAU |
| has-miR-30e-5p | UGUAAACAUCCUUGACUGGAAG |
| has-miR-375 | UUUGUUCGUUCGGCUCGCGUGA |
| has-miR-378a-3p | ACUGGACUUGGAGUCAGAAGGC |
| has-miR-151a-3p | CUAGACUGAAGCUCCUUGAGG |
| has-miR-425-5p | AAUGACACGAUCACUCCCGUUGA |
| has-miR-484 | UCAGGCUCAGUCCCCUCCCGAU |
| has-miR-146b-5p | UGAGAACUGAAUUCCAUAGGCU |
| has-miR-574-5p | UGAGUGUGUGUGUGUGAGUGUGU |
| has-miR-652-3p | AAUGGCGCCACUAGGGUUGUG |
| has-miR-320c | AAAAGCUGGGUUGAGAGGGU |
| has-miR-3141 | GAGGGCGGGUGGAGGAGGA |

### Example 2—In Vitro Evaluation of miR-3184, miR-3126, and miR-3141

Sample Processing

Cell Culture and Transfection

Caco2 were cultured in DMEM (+) L-glutamate (−) Pyruvate supplemented with 1× non-essential amino acids, 1 mM Sodium Pyruvate and 20% FBS. Cells were split three times a week without exciding 80% confluency. For transfection, cells were plated in 24 well plate (50,000 cells/well) to obtain a 50% confluency the day after. Transfection was performed using DharmaFect 4 reagent from Dharmacon (cat #T-2004-01) following manufacturer recommendation. Briefly, 1.25 ul/well of DharmaFect 4 and a final concentration of 25 nM of miRNA were used. For each time point (i.e. 48 h and 72 h post transfection) media was removed and cells were washed with 1 ml of cold PBS without calcium chloride and magnesium chloride. Cells were frozen directly on plate after aspirating cold PBS.

miRNA were purchased from Dharmacon (Horizon Discovery), see table below.

| cat # | Name |
|---|---|
| C-301683-00-0020 | miRIDIAN microRNA Human hsa-miR-3141 - |
| | Mimic |
| C-301661-00-0020 | miRIDIAN microRNA Human hsa-miR-3126-5p - |
| | Mimic |
| C-302745-00-0020 | miRIDIAN microRNA Human hsa-miR-3184-3p - |
| | Mimic |
| C-301151-01-0020 | miRIDIAN microRNA Human hsa-miR-149-3p - |
| | Mimic |
| C-301113-01-0020 | miRIDIAN microRNA Human hsa-miR-574-5p - |
| | Mimic |
| CN-002000-01-20 | miRIDIAN microRNA Mimic Negative Control #2 |
| CP-004000-01-20 | Mimic Endogenous Positive Control (miR-122) |

RNA Extraction

RNA were extracted using the QIAymphony (from QIAGEN) robot and the RNA kit extraction (QIAGEN cat #931636) with the miRNA CT400 protocol. Cells were lyzed into 420 ul of RLT plus buffer and RNA was eluted into 100 ul. RNA quality and quantity were assessed using the Fragment Analyzer and the RiboGreen technology respectively.

QuantSeq Experiment and Sequencing

A starting material of 50 ng (RNA) was used for the QuantSeq protocol following manufacturer recommendations (cat #: 015.384 Lexogen). Libraries were sequenced single-end for 65 bp (SR50) on an Illumina HiSeq 2500 instrument using a High output SBS V4 kit. Samples were sequenced to a depth of 6-10 million reads.

Data Analysis

Gene expression levels were filtered prior to normalization and differential expression analysis. Genes with count per million of reads (CPM) below 19.89 in at least 9 samples were discarded. These genes were considered lowly expressed and below the level of detection. The gene expression levels for the remaining genes were then normalized. The normalization was done with the weighted trimmed means of the log expression ratios (TMM) method as described in Robinson, and Oshlack, Genome Biology 11, 2010. In order to estimate the differentially expressed genes, samples were grouped by treatment and time, and expression values were modeled with a quasi-likelihood negative binomial generalized log-linear model to count data as described in Lund, and Nettleton, Statistical Applications in Genetics and Molecular Biology. 2012. Model parameters were set as below:

Expgene=group+batch

Eventually multiple testing procedure and significance level cutoff to the genewise tests was performed to prevent false positive rate inflation due to the multiplication of the statistical tests performed.

Biological Function and Pathway Analysis with Ingenuity Pathway Analysis (IPA) Software

Functional enrichment analysis was performed using Ingenuity Pathway Analysis (IPA) software. The analysis examines genes in the dataset that are known to affect functions, or pathways, compares the genes' direction of change to expectations derived from the literature, then issues a prediction for each function or pathway based on the direction of change. The direction of change is the gene expression in the experimental samples relative to a control. If the direction of change is consistent with the literature across most genes, IPA predicts that the function or pathway will increase in the experimental sample. If it is mostly inconsistent with the literature, IPA predicts that the function or pathway will decrease. If there is no clear pattern related to the literature, IPA does not make any prediction

IPA uses the z-score algorithm to make predictions. The z-score algorithm is designed to reduce the chance that random data will generate significant predictions. See Kramer A. et al., Bioinformatics, 2014 for a detailed description of the algorithm.

Results

An in vitro Caco2 cell model was used to evaluate the effect of miR-3184-3p, miR-3126-5p, and miR-3141 on gene expression.

miR-3184-3p, miR-3126-5p, and miR-3141 significantly effected the expression of the genes shown in Table 4 below.

TABLE 4
in vitro evaluation of miR-3184, miR-3126, and miR-3141

| | Gene expression log fold change |
|---|---|
| miRNA human ID | miR treated cells versus control cells |
| miR-3184-3p | Lipin 2 +1.54 |
| | IGF2 +1.72 |
| miR-3126-5p | Ninein +1.93 |
| | Unc-13 homolog D +2.46 |
| | Sulfatase 2 +2.29 |
| | IL-32 +1.69 |
| | OXER1 −2.16 |
| | TMEM127 −3.14 |
| | Butyrophilin +2.24 |
| | ITGA2 +2.57 |
| | ITGB1 +1.28 |
| | TUBA4A +2.43 |
| | TUBB6 +0.91 |
| | TUBB2A +2.17 |
| | TUBB2B +2.31 |
| miR-3141 | Claudin-2 +4.35 |
| | SLC2A1 +1.65 |

Modulation of pathways and biological functions was found analysing the differentially expressed genes in the Caco2 model in Ingenuity Pathway Analysis software following the procedure described here above (Biological function and pathway analysis with Ingenuity Pathway analysis (IPA) software). In terms of pathway modulation, the results of this analysis are as follows:

miR3126-5p modulates PI3K/AKT signalling, AHR signalling and epithelial adherens junction pathway.

miR-3141 modulates TR/RXR pathway and RXR pathway.

miR-3184-3p modulates antiproliferative role of TOB in T cell signaling.

All publications mentioned in the above specification are herein incorporated by reference.

Various modifications and variations of the disclosed methods, cells, compositions and uses of the invention will be apparent to the skilled person without departing from the scope and spirit of the invention. Although the invention has been disclosed in connection with specific preferred embodiments, it should be understood that the invention as claimed should not be unduly limited to such specific embodiments. Indeed, various modifications of the disclosed modes for carrying out the invention, which are obvious to the skilled person are intended to be within the scope of the following claims.

1-16. (canceled)

17. A nutritional composition comprising miR-3184.

18. A nutritional composition according to claim 17, wherein the nutritional composition is in a form selected from the group consisting of an infant formula, a fortifier, and a supplement.

19. A nutritional composition according to claim 17, wherein the nutritional composition is an infant formula.

20. A nutritional composition according to claim 17, wherein the miR-3184 is present in a concentration of 0.1-10000 pmol/L.

21. A nutritional composition according to claim 17, wherein the nutritional composition comprises one or more additional microRNAs selected from the group consisting of: let-7b, let-7c, miR-19b, miR-22, miR-24, miR-25, miR-29a, miR-30a, miR-92a, miR-99a, miR-100, miR-197, miR-30d, miR-181a, miR-181b, miR-205, miR-210, miR-221, miR-125b, miR-125a, miR-149, miR-193a, miR-320a, miR-200a, miR-99b, miR-130b, miR-30e, miR-375, miR-378a, miR-151a, miR-425, miR-484, miR-146b, miR-574, miR-652, miR-320c, miR-3141, let-7d, miR-196a, miR-187, miR-516a, miR-92b, and miR-3126.

22. A nutritional composition according to claim 17, wherein the nutritional composition comprises one or more additional microRNAs selected from the group consisting of: let-7d, miR-196a, miR-187, miR-516a, miR-92b, and miR-3126.

23. A nutritional composition according to claim 17, wherein the nutritional composition comprises miR-3141 and/or miR-3126.

24. A method of promoting healthy growth and development and preventing metabolic disorders later in life in a subject comprising administering to the subject a nutritional composition comprising miR-3184.

25. A method according to claim 24, wherein the composition is administered to an infant.

26. A method to modulate gene expression of one or more gene selected from: Lipin 2 or Insulin Like Growth Factor 2 comprising using miR-3184.


SEQUENCE LISTING (90 sequences)

SEQ ID NO: 1 - RNA, 75 nt, Homo sapiens
aagcaagacugaggggccucagaccgagcuuuuggaaaauagaaaagucucgcucucugccccucagccuaacuu

SEQ ID NO: 2 - RNA, 24 nt, Homo sapiens
ugaggggccucagaccgagcuuuu

SEQ ID NO: 3 - RNA, 23 nt, Homo sapiens
aaagucucgcucucugccccuca

SEQ ID NO: 4 - RNA, 22 nt, Homo sapiens
  let
ugagguaguagguugugugguu

SEQ ID NO: 5 - RNA, 22 nt, Homo sapiens
  let
ugagguaguagguuguaugguu

SEQ ID NO: 6 - RNA, 23 nt, Homo sapiens
ugugcaaauccaugcaaaacuga

SEQ ID NO: 7 - RNA, 22 nt, Homo sapiens
aagcugccaguugaagaacugu

SEQ ID NO: 8 - RNA, 22 nt, Homo sapiens
uggcucaguucagcaggaacag

SEQ ID NO: 9 - RNA, 22 nt, Homo sapiens
cauugcacuugucucggucuga

SEQ ID NO: 10 - RNA, 22 nt, Homo sapiens
uagcaccaucugaaaucgguua

SEQ ID NO: 11 - RNA, 22 nt, Homo sapiens
uguaaacauccucgacuggaag

SEQ ID NO: 12 - RNA, 22 nt, Homo sapiens
uauugcacuugucccggccugu

SEQ ID NO: 13 - RNA, 22 nt, Homo sapiens
aacccguagauccgaucuugug

SEQ ID NO: 14 - RNA, 22 nt, Homo sapiens
aacccguagauccgaacuugug

SEQ ID NO: 15 - RNA, 22 nt, Homo sapiens
uucaccaccuucuccacccagc

SEQ ID NO: 16 - RNA, 22 nt, Homo sapiens
uguaaacauccccgacuggaag

SEQ ID NO: 17 - RNA, 23 nt, Homo sapiens
aacauucaacgcugucggugagu

SEQ ID NO: 18 - RNA, 23 nt, Homo sapiens
aacauucauugcugucggugggu

SEQ ID NO: 19 - RNA, 22 nt, Homo sapiens
uccuucauuccaccggagucug

SEQ ID NO: 20 - RNA, 22 nt, Homo sapiens
cugugcgugugacagcggcuga

SEQ ID NO: 21 - RNA, 23 nt, Homo sapiens
agcuacauugucugcuggguuuc

SEQ ID NO: 22 - RNA, 22 nt, Homo sapiens
ucccugagacccuaacuuguga

SEQ ID NO: 23 - RNA, 24 nt, Homo sapiens
ucccugagacccuuuaaccuguga

SEQ ID NO: 24 - RNA, 21 nt, Homo sapiens
agggagggacgggggcugugc

SEQ ID NO: 25 - RNA, 22 nt, Homo sapiens
ugggucuuugcgggcgagauga

SEQ ID NO: 26 - RNA, 22 nt, Homo sapiens
aacuggccuacaaagucccagu

SEQ ID NO: 27 - RNA, 22 nt, Homo sapiens
aaaagcuggguugagagggcga

SEQ ID NO: 28 - RNA, 22 nt, Homo sapiens
uaacacugucugguaacgaugu

SEQ ID NO: 29 - RNA, 22 nt, Homo sapiens
cacccguagaaccgaccuugcg

SEQ ID NO: 30 - RNA, 22 nt, Homo sapiens
cagugcaaugaugaaagggcau

SEQ ID NO: 31 - RNA, 22 nt, Homo sapiens
uguaaacauccuugacuggaag

SEQ ID NO: 32 - RNA, 22 nt, Homo sapiens
uuuguucguucggcucgcguga

SEQ ID NO: 33 - RNA, 22 nt, Homo sapiens
acuggacuuggagucagaaggc

SEQ ID NO: 34 - RNA, 21 nt, Homo sapiens
cuagacugaagcuccuugagg

SEQ ID NO: 35 - RNA, 23 nt, Homo sapiens
aaugacacgaucacucccguuga

SEQ ID NO: 36 - RNA, 22 nt, Homo sapiens
ucaggcucaguccccucccgau

SEQ ID NO: 37 - RNA, 22 nt, Homo sapiens
ugagaacugaauuccauaggcu

SEQ ID NO: 38 - RNA, 23 nt, Homo sapiens
ugagugugugugugugagugugu

SEQ ID NO: 39 - RNA, 21 nt, Homo sapiens
aauggcgccacuaggguugug

SEQ ID NO: 40 - RNA, 20 nt, Homo sapiens
aaaagcuggguugagagggu

SEQ ID NO: 41 - RNA, 19 nt, Homo sapiens
gagggcggguggaggagga

SEQ ID NO: 42 - RNA, 17 nt, Homo sapiens
gaggcugagcugaggag

SEQ ID NO: 43 - RNA, 22 nt, Homo sapiens
cuauacgaccugcugccuuucu

SEQ ID NO: 44 - RNA, 22 nt, Homo sapiens
uagguaguuucauguuguuggg

SEQ ID NO: 45 - RNA, 22 nt, Homo sapiens
ggcuacaacacaggacccgggc

SEQ ID NO: 46 - RNA, 23 nt, Homo sapiens
uucucgaggaaagaagcacuuuc

SEQ ID NO: 47 - RNA, 22 nt, Homo sapiens
agggacgggacgcggugcagug

SEQ ID NO: 48 - RNA, 22 nt, Homo sapiens
ugagggacagaugccagaagca

SEQ ID NO: 49 - RNA, 22 nt, Bos taurus
  let
ugagguaguagguugugugguu

SEQ ID NO: 50 - RNA, 22 nt, Bos taurus
  let
ugagguaguagguuguaugguu

SEQ ID NO: 51 - RNA, 23 nt, Bos taurus
ugugcaaauccaugcaaaacuga

SEQ ID NO: 52 - RNA, 21 nt, Bos taurus
aagcugccaguugaagaacug

SEQ ID NO: 53 - RNA, 22 nt, Bos taurus
uggcucaguucagcaggaacag

SEQ ID NO: 54 - RNA, 22 nt, Bos taurus
cauugcacuugucucggucuga

SEQ ID NO: 55 - RNA, 23 nt, Bos taurus
cuagcaccaucugaaaucgguua

SEQ ID NO: 56 - RNA, 24 nt, Bos taurus
uguaaacauccucgacuggaagcu

SEQ ID NO: 57 - RNA, 22 nt, Bos taurus
uauugcacuugucccggccugu

SEQ ID NO: 58 - RNA, 21 nt, Bos taurus
aacccguagauccgaucuugu

SEQ ID NO: 59 - RNA, 22 nt, Bos taurus
aacccguagauccgaacuugug

SEQ ID NO: 60 - RNA, 22 nt, Bos taurus
uucaccaccuucuccacccagc

SEQ ID NO: 61 - RNA, 24 nt, Bos taurus
uguaaacauccccgacuggaagcu

SEQ ID NO: 62 - RNA, 24 nt, Bos taurus
aacauucaacgcugucggugaguu

SEQ ID NO: 63 - RNA, 24 nt, Bos taurus
aacauucauugcugucgguggguu

SEQ ID NO: 64 - RNA, 22 nt, Bos taurus
uccuucauuccaccggagucug

SEQ ID NO: 65 - RNA, 23 nt, Bos taurus
acugugcgugugacagcggcuga

SEQ ID NO: 66 - RNA, 22 nt, Bos taurus
agcuacauugucugcuggguuu

SEQ ID NO: 67 - RNA, 22 nt, Bos taurus
ucccugagacccuaacuuguga

SEQ ID NO: 68 - RNA, 23 nt, Bos taurus
ucccugagacccuuuaaccugug

SEQ ID NO: 69 - RNA, 22 nt, Bos taurus
gagggagggacgggggcugugc

SEQ ID NO: 70 - RNA, 22 nt, Bos taurus
ugggucuuugcgggcgagauga

SEQ ID NO: 71 - RNA, 22 nt, Bos taurus
aacuggccuacaaagucccagu

SEQ ID NO: 72 - RNA, 22 nt, Bos taurus
aaaagcuggguugagagggcga

SEQ ID NO: 73 - RNA, 23 nt, Bos taurus
uaacacugucugguaacgauguu

SEQ ID NO: 74 - RNA, 22 nt, Bos taurus
cacccguagaaccgaccuugcg

SEQ ID NO: 75 - RNA, 22 nt, Bos taurus
cagugcaaugaugaaagggcau

SEQ ID NO: 76 - RNA, 24 nt, Bos taurus
uguaaacauccuugacuggaagcu

SEQ ID NO: 77 - RNA, 23 nt, Bos taurus
uuuuguucguucggcucgcguga

SEQ ID NO: 78 - RNA, 22 nt, Bos taurus
acuggacuuggagucagaaggc

SEQ ID NO: 79 - RNA, 21 nt, Bos taurus
cuagacugaagcuccuugagg

SEQ ID NO: 80 - RNA, 22 nt, Bos taurus
augacacgaucacucccguuga

SEQ ID NO: 81 - RNA, 22 nt, Bos taurus
ucaggcucaguccccucccgau

SEQ ID NO: 82 - RNA, 24 nt, Bos taurus
ugagaacugaauuccauaggcugu

SEQ ID NO: 83 - RNA, 24 nt, Bos taurus
ugagugugugugugugagugugug

SEQ ID NO: 84 - RNA, 21 nt, Bos taurus
aauggcgccacuaggguugug

SEQ ID NO: 85 - RNA, 20 nt, Bos taurus
agcuggguugagaggguggu

SEQ ID NO: 86 - RNA, 18 nt, Bos taurus
gagggcggguggaggagg

SEQ ID NO: 87 - RNA, 22 nt, Bos taurus
  let
agagguaguagguugcauaguu

SEQ ID NO: 88 - RNA, 22 nt, Bos taurus
uagguaguuucauguuguuggg

SEQ ID NO: 89 - RNA, 22 nt, Bos taurus
ucgugucuuguguugcagccgg

SEQ ID NO: 90 - RNA, 22 nt, Bos taurus
uauugcacucgucccggccucc